(19) United States
(12) Patent Application Publication
(10) Pub. No.: US 2026/0250223 A1
(43) Pub. Date: Aug. 27, 2026

(54) METHOD AND APPARATUS FOR PRODUCTION OF ALCOHOLS

(71) Applicant: JOHNSON MATTHEY DAVY TECHNOLOGIES LIMITED, London (GB)

(72) Inventor: Martin SMIDT, London (GB)

(21) Appl. No.: 18/855,201

(22) PCT Filed: Jun. 6, 2023
(86) PCT No.: PCT/GB2023/051472

§ 371 Date: Oct. 8, 2024

(30) Foreign Application Priority Data
Jun. 7, 2022 (GB) 2208331.5

(51) Int. Cl. C07C 29/80; B01D 3/14; B01J 8/04; C07C 29/141; C07C 45/50

(52) U.S. Cl. CPC C07C 29/80; B01D 3/143; B01J 8/0492; C07C 29/141; C07C 45/50

(57) ABSTRACT

A method for the production of at least one alcohol is provided. The method comprises providing a crude aldehyde stream comprising at least one aldehyde to at least one liquid phase hydrogenation reactor, and hydrogenating the at least one aldehyde in a liquid phase hydrogenation reaction to provide a crude alcohol stream comprising at least one alcohol; recycling a liquid recycle stream comprising the at least one alcohol to the at least one liquid hydrogenation reactor via a recycle cooler in which heat in the liquid recycle stream is recovered; providing the crude alcohol stream to a distillation column and performing distillation on the crude alcohol stream to obtain a purified alcohol stream including the at least one alcohol, and a heavy stream including the at least one alcohol to be processed into a recycling stream and a waste stream.

The present invention relates to a method and apparatus for the production of alcohols. In particular, the methods and apparatus according to the present invention provide improved yield of synthetic alcohols.

### BACKGROUND

Alcohols, particularly C₁-C₁₀ alcohols, are used in a wide variety of applications in the chemical industries including as solvents, fuels, chemical intermediates in synthesis of organic compounds, and such like. Globally, a wide variety of alcohols are produced each year. For certain alcohols (e.g. ethanol) biosynthetic methods such as fermentation may be suitable for the production of alcohol. However, for longer chain and branched-chain alcohols, synthetic production may be required.

Existing methods for the synthetic production of alcohols include the steps of hydroformylation of olefins in the presence of a catalyst (also referred to as the ‘Oxo’ process), followed by a hydrogenation reaction. The advantages of such methods are cheap small-molecule organic starting materials.

Hydroformylation introduces a formyl group to the unsaturated olefin to provide an aldehyde. Hydroformylation may be effected by contacting the olefin with syngas (a mixture of carbon monoxide and hydrogen). Once the aldehyde has been produced, hydrogenation reduces the aldehyde to furnish the corresponding alcohol. Where longer chain alcohols are required, the process may include the step of aldolization to condense two aldehydes, thus providing a longer chain unsaturated aldehyde. The longer chain unsaturated aldehyde is then subsequently reduced by hydrogenation to provide the corresponding saturated alcohol.

Following hydrogenation, the reaction effluent comprises the alcohol product in a crude mixture i.e. a crude alcohol mixture. The crude alcohol mixture is subjected to a distillation step to isolate and purify the alcohol. However, the requirement for high product purity (i.e. high purity alcohol) means that the purified alcohol fraction has a narrow cut point (i.e. a narrow boiling point range). As such, a significant amount of alcohol may be disposed of as waste. In particular, a significant amount of alcohol may remain in the higher boiling point fraction, referred to in the art as the ‘heavy stream’, which is typically disposed of as a waste stream. If the distillation is performed so as to recover all the alcohol as product (i.e. the purified alcohol fraction has a wider boiling point range), then contaminants (e.g. heavy chain hydrocarbons) will be present in the purified alcohol. Contaminants may include by-products of the hydroformylation and hydrogenation reactions (e.g. unwanted oxidation and condensation products such as esters, ketones and other oxygenates). To mitigate contamination of the purified alcohol product, the distillation is performed such that some alcohol remains in the heavy stream. Performing the distillation such that some alcohol remains in the heavy stream may also be beneficial in order to prevent excessive temperatures in the bottom of distillation columns. Such excessive temperatures can lead to decomposition of heavy components into lighter components that can travel back up the distillation column and contaminate the product.

As significant amounts of alcohol product are disposed of, existing methods for the production of alcohols are inefficient.

Prior art methods may dispose of the heavy stream, or alternatively subject the heavy stream to further processing or refining steps.

U.S. Pat. No. 5,004,845 describes a heavy stream comprising butanol, which is mixed with hydrogen, preheated and vaporised in a vaporiser. The resulting vapour mixture is then directed to a vapour phase hydrogenation (VPH) reactor. As the whole heavy stream is vaporised, contaminants may accumulate in the process which can adversely affect the performance of the hydroformylation and hydrogenation catalysts, as well as the quality and purity of the alcohol product. CN107032953 also describes a vapour phase hydrogenation (VPH) reaction scheme. Hydrogenation may be used to crack heavier chain hydrocarbon contaminants. For example, Butyl butyrate (BuBu), is a common by-product of butanal hydrogenation, which can be hydrogenated to provide butanol.

Other methods treat the heavy stream in a column operating at reduced pressure and maintaining a low temperature to allow for removal of lighter components without causing the heavier components to decompose. Alternatively, the column may be operated at higher temperatures, or additives (e.g. hydroxides) provided to promote the heavier contaminant compounds to decompose to lighter compounds such as alcohols and aldehydes. DE2713434 describes separating higher-boiling point fractions in a column.

Other methods treat the heavy stream in a steam stripper, wherein the lighter compounds are separated from the heavy compounds using steam. The product stream may contain the desired alcohol and may also contain aldehydes or other components. The water is removed from the product stream, and the resulting lights stream is directed to a hydrogenation reactor or to a lights column for further separation. U.S. Pat. No. 2,614,128 describes recovering alcohol from a heavy stream in a steam stripper and recovering the alcohol for recycle to the hydrogenation stage of the Oxo reaction.

Recovering alcohol from the heavy stream with any of the aforementioned methods requires additional equipment, increased energy costs, and may cause accumulation of contaminants in the process.

Some prior art vapour phase hydrogenation systems have used a hydrogen gas stream as a strip gas to strip alcohol from an alcohol containing heavy stream before passing the resulting alcohol-containing hydrogen gas stream to the vapour phase hydrogenation. However, such systems may not be cost-effective as there is already a vaporiser present to vaporise the aldehyde feed to the vapour phase hydrogenation reaction, and to which the heavy stream could be passed to vaporise alcohol from the heavy stream, and the addition of another stripper may therefore increase equipment count for no overall benefit.

It is an object of the present invention to obviate or mitigate one or more of the aforementioned problems.

### SUMMARY OF INVENTION

The present invention relates to improvements in the overall yield and purity of alcohol obtained from a process for the production of alcohols. In particular, the present invention recovers increased quantities of alcohol from the effluent of a hydroformylation and hydrogenation process. The present invention advantageously recovers alcohol from a heavy stream using a stripping gas comprising hydrogen, where the heavy stream is obtained from the distillation of alcohol from a crude alcohol stream. The resulting recycled stream comprises both the alcohol recovered from the heavy stream and the stripping gas, and can be used directly in a liquid phase hydrogenation reaction. The method and apparatus disclosed herein therefore advantageously provides (i) increased alcohol yield by recovering alcohol from the heavy stream and returning alcohol to the process; (ii) a recycle stream that can be directly used in a liquid phase hydrogenation reaction.

According to a first aspect of the present invention, there is provided a method for the production of at least one alcohol comprising the steps of (i) providing a crude aldehyde stream comprising at least one aldehyde to at least one liquid phase hydrogenation reactor, and hydrogenating the at least one aldehyde in a liquid phase hydrogenation reaction to provide a crude alcohol stream comprising at least one alcohol; (ii) recycling a liquid recycle stream comprising the at least one alcohol to the at least one liquid hydrogenation reactor via a recycle cooler in which heat in the liquid recycle stream is recovered, preferably by raising steam, (iii) providing the crude alcohol stream to a distillation column and performing distillation on the crude alcohol stream to provide a purified alcohol stream including the at least one alcohol, and a heavy stream including the at least one alcohol; (iv) providing the heavy stream to a stripping column and contacting the heavy stream with a stripping gas comprising at least 20 mol % hydrogen to separate the heavy stream into a recycle stream comprising the at least one alcohol, and a waste stream; and (v) returning at least a portion of the recycle stream to the at least one liquid phase hydrogenation reactor in step (i).

In embodiments, the at least one alcohol is selected from at least one C₃-C₂₀ alcohol, optionally at least one C₃-C₁₀ alcohol, optionally still at least one C₄-C₁₀ alcohol. In embodiments, the at least one alcohol is selected from butanol, 2-ethyhexanol, 2-propyl heptanol and isononyl-alcohol.

In embodiments, a reactor outlet stream is split to provide the crude alcohol stream and the liquid recycle stream. In alternative embodiments the liquid recycle stream and the crude alcohol stream are recovered separately from the at least one liquid phase hydrogenation reactor.

In embodiments, the crude alcohol stream comprises at least 50 wt % of the at least one alcohol, optionally at least 60 wt % of the at least one alcohol, optionally still at least 70 wt % of the at least one alcohol.

In embodiments, the purified alcohol stream comprises greater than 95 wt % of the at least one alcohol, optionally greater than 97 wt % of the at least one alcohol, and optionally still greater than 99 wt % of the at least one alcohol, relative to the total weight of the purified alcohol stream.

In embodiments, the heavy stream comprises less than 80 wt % of the at least one alcohol, optionally less than 70 wt % of the at least one alcohol, optionally less than 60 wt % of the at least one alcohol. Preferably, the heavy stream comprises at least 40 wt %, preferably at least 50 wt % of the at least one alcohol. An advantage of the invention is that high alcohol percentages in the heavy stream can be tolerated because that alcohol is being recovered. Higher alcohol percentages in the heavy stream may permit more efficient operation of the distillation while maintaining alcohol product quality, for example because of reduced cracking of heavy components in the bottom of distillation columns due to the consequent lower temperatures.

Another advantage of the invention in liquid phase hydrogenation reactions is that the alcohol recovered from the heavy stream is condensed in the at least one liquid phase hydrogenation reactor and thus introduces energy into the at least one liquid phase hydrogenation reactor. This energy is then recovered in the recycle cooler, for example in the form of extra steam raised in the recycle cooler. The combination, in a liquid phase hydrogenation reaction, of the hydrogen stripper to recover the alcohol from the heavy stream through vaporisation and the recycling a liquid recycle stream via a recycle cooler in which heat is recovered thus results in particularly energy efficient operation. Prior art vapour phase hydrogenation reactor systems do not benefit from this advantage. The recycle cooler may recover heat by raising steam. It will be appreciated that recovered heat can be used elsewhere in the plant or process.

In embodiments, the recycle stream comprises from 1 to 25 wt % of the at least one alcohol, optionally from 5 to 20 wt % of the at least one alcohol, optionally from 10-15 wt % of the at least one alcohol.

In embodiments, the stripping gas stream comprises at least 70 mol % hydrogen, optionally at least 90 mol % hydrogen, optionally still at least 98 mol % hydrogen. In embodiments, the recycle stream comprises at least 60 wt % hydrogen, optionally at least 70 wt % hydrogen, optionally still at least 80 wt % hydrogen. In embodiments, the recycle stream comprises at least 2 wt % alcohol, optionally at least 5 wt % alcohol, optionally still at least 7 wt % alcohol. In embodiments, the recycle stream comprises at least 70 wt % hydrogen and at least 5 wt % alcohol.

In embodiments, the pressure and temperature in the stripping column is selected to strip the at least one alcohol from the heavy stream, whilst heavier compounds remain in the heavy stream. In embodiments, the temperature and pressure inside the stripping column are such that the heavy stream remains in the liquid phase, and the stripping gas remains in the gaseous phase. In embodiments, the pressure in the stripping column is from 15-45 bara, optionally from 20-35 bara. The temperatures and pressures in the stripping column may be controlled to keep the temperature in the liquid outlet above a minimum value. The liquid outlet may be chilled by the vaporisation of the alcohol into the stripping gas and so the liquid outlet may represent the minimum temperature in the stripping column. In embodiments, the temperature of the liquid outlet from the stripping column is not less than 40° C. and preferably not less than 50° C. Lower temperatures may result in the outlet being too viscous or poor recovery of the alcohol.

In embodiments, (i) the stripping gas stream is provided to a lower portion of the stripping column, optionally to the bottom of the stripping column; and (ii) the heavy stream is provided to an upper portion of the stripping column, optionally to the top of the stripping column. In embodiments, the recycle stream is obtained from an upper portion of the stripping column, optionally from the top of the stripping column. The heavy stream descends down the stripping column under the influence of gravity, and the stripping stream ascends up the stripping column being less dense that the heavy stream. As the heavy stream and stripping gas contact each other, volatile molecules such as the at least one alcohol move from the liquid phase (heavy stream) to the vapour phase (stripping gas) and thus are “stripped” from the heavy stream.

In embodiments, the temperature of the stripping gas stream entering the stripping column is from 50-250° C., optionally from 60-200° C. In embodiments, the temperature of the heavy stream entering the stripping column is from 50-300° C., optionally, from 100-200° C., optionally still from 125-175° C. (e.g. around 130-160° C.).

In embodiments, the stripping gas stream may comprise at least 50 wt %, optionally at least 70 wt % and optionally still at least 90 wt % of the hydrogen fed to the liquid phase hydrogenation reactor. Preferably the stripping gas stream is the sole source of hydrogen feed to the liquid phase hydrogenation reactor. In other words, the hydrogen required for the hydrogenation of the aldehyde in the liquid phase hydrogenation reactor is provided in the stripping gas stream and not in a separate hydrogen feed stream to the liquid phase hydrogenation reactor. Using a larger flowrate of hydrogen in the stripping gas may advantageously result in lower temperatures in the stripping column and it may thus be beneficial to include all the hydrogen required for the liquid phase hydrogenation reaction in the stripping gas stream.

In embodiments, the step of providing a crude aldehyde stream comprises the steps of providing an olefin stream to at least one hydroformylation reactor, and contacting the olefin stream with a hydroformylation gas stream comprising hydrogen and carbon monoxide to effect a hydroformylation reaction to provide the crude aldehyde stream.

In embodiments, the crude aldehyde stream comprises at least one C₃-C₂₀ aldehyde, optionally at least one C₃-C₁₀ aldehyde, optionally still at least one C₄-C₁₀ aldehyde. In embodiments, the at least one aldehyde is selected from butanal, 2-ethyl hexenal, 2-propyl heptenal and iso nonanal. In embodiments, the crude aldehyde stream comprises at least 40 wt % aldehyde, optionally at least 50 wt % aldehyde, optionally still at least 60 wt % aldehyde.

In embodiments, providing the crude aldehyde stream further comprises subjecting, for example by contacting the reaction effluent with at least one base or at least one acid, a reaction effluent of the hydroformylation reaction to an aldol reaction between at least two aldehydes comprised in the reaction effluent, to provide the crude aldehyde stream. In such embodiments, the crude aldehyde stream comprises at least one aldol condensation product. In such embodiments, the crude aldehyde stream may comprise at least 20 wt % aldol condensation product, optionally at least 30 wt % aldol condensation product, optionally still at least 40 wt % aldol condensation product. In preferred embodiments, the crude aldehyde stream comprises at least one unsaturated aldehyde, for example 2-ethyl hexenal or 2-propyl heptenal. 2-ethyhexenal is the aldol condensation product of butanal. 2-propyl heptenal is the aldol condensation product of pentanal. In embodiments, butanal and pentanal may be obtained from hydroformylation of the olefins propene and butene respectively.

In embodiments, prior to step (v) the recycle stream is directed to a secondary hydrogenation reactor configured to hydrogenate esters into at least one alcohol, wherein the recycle stream is contacted with hydrogen to effect a hydrogenation reaction. In embodiments, the hydrogenation reaction in the secondary hydrogenation reactor is a vapour phase hydrogenation reaction. In embodiments, the hydrogenation reaction in the secondary hydrogenation reactor is a liquid phase hydrogenation reaction. The esters may be comprised in the heavy stream and be stripped from the heavy stream by the stripping gas along with the alcohol. Stripping those esters from the heavy stream in accordance with the present invention and hydrogenating them to the corresponding alcohol may advantageously increase the overall alcohol yield of the liquid phase hydrogenation and thus improve the feedstock efficiency of the whole process.

According to a second aspect of the present invention, there is provided apparatus for the production of alcohol comprising at least one liquid phase hydrogenation reactor configured to hydrogenate at least one aldehyde comprised in a crude aldehyde stream in a liquid phase hydrogenation reaction to provide a crude alcohol stream comprising at least one alcohol; a recycle loop in fluid communication with the at least one liquid phase hydrogenation reactor and a recycle cooler, the recycle loop being configured to recycle a liquid recycle stream from the at least one liquid phase hydrogenation reactor via the recycle cooler to the at least one liquid phase hydrogenation reactor, a distillation column in fluid communication with the at least one liquid phase hydrogenation reactor, the distillation column being configured to perform distillation on the crude alcohol stream to provide a purified alcohol stream comprising the at least one alcohol, and a heavy stream comprising the at least one alcohol; a stripping column in fluid communication with the distillation column, the stripping column being configured to contact said heavy stream with a stripping gas stream comprising at least 20 mol % hydrogen to separate the heavy stream into a recycle stream comprising the at least one alcohol, and a waste stream; wherein the stripping column is in fluid communication with the at least one liquid phase hydrogenation reactor to provide the recycle stream to the at least one liquid phase hydrogenation reactor.

In embodiments, the stripping column is a hydrogen stripping column, that is to say hydrogen is used as the stripping gas.

In embodiments, the apparatus further comprises a hydroformylation reactor configured to contact an olefin stream comprising at least one olefin with hydrogen and carbon monoxide to effect a hydroformylation reaction on the at least one olefin, and optionally an aldolization unit to effect an aldol condensation reaction to convert aldehydes produced in the hydroformylation reaction to longer chain unsaturated aldehydes, to provide the crude aldehyde stream.

In embodiments, at least one olefin comprised in the olefin stream is propene, the at least one aldehyde comprised in the crude aldehyde stream is butanal and/or 2-ethyl hexenal, and the at least one alcohol comprised in the crude alcohol stream is butanol and/or 2-ethyl hexanol.

In embodiments, at least one olefin comprised in the olefin stream is butylene, the at least one aldehyde comprised in the crude aldehyde stream is pentanal and/or 2-propyl heptenal, and the at least one alcohol comprised in the crude alcohol stream is pentanol and/or 2-propyl heptanol.

### BRIEF DESCRIPTION OF FIGURES

FIG. 1A illustrates a schematic of a prior art method for the production of alcohol.

FIG. 1B illustrates a schematic of an apparatus suitable for implementing the method of FIG. 1A.

FIG. 2A illustrates a schematic of a method for the production of alcohol according to the present invention.

FIG. 2B illustrates a schematic of an apparatus suitable for implementing the method of FIG. 2A.

FIG. 3A illustrates a schematic of a method for the production of alcohol according to the present invention.

FIG. 3B illustrates a schematic of an apparatus suitable for implementing the method of FIG. 3A.

FIG. 4 illustrates a schematic of an apparatus suitable for implementing a method of producing alcohol according to the present invention.

### DETAILED DESCRIPTION

The present invention will now be described, by way of example only, with reference to the accompanying drawings and examples. The scope of protection is defined in the appended claims.

“Alcohol” as used herein refers to linear or branched C₃-C₂₀ alcohols. The alcohols produced in accordance with the present invention may be mono-alcohols (i.e. comprise one hydroxyl group) or polyols (i.e. comprise multiple hydroxyl groups). Polyols may comprises 2-8 hydroxyl groups, and preferably comprise 2 hydroxyl groups. In embodiments, the alcohol produced is a monohydroxylated linear or branched C₃-C₂₀ alcohols. In embodiments, the alcohol produced is selected from propanol, butanol, pentanol, hexanol, heptanol, octanol, nonanol, decanol, and isomers thereof. In embodiments, the alcohol produced is selected from butanol, 2-ethyl hexanol and 2-propyl heptanol. 2-ethyl hexanol is widely used to make plasticiser alcohols, such as bis-2ethylhexyl-phthalate.

The term “crude” as used herein means that the ‘crude’ stream has not been purified, and contains a mixture of compounds. The ‘crude’ stream thus requires further processing and/or purification to provide the final product (i.e. alcohol). The “crude” streams described herein may therefore be the effluent from a reaction process (e.g. the effluent from a hydrogenation reaction, and/or the effluent from a hydroformylation reaction). As such, the crude stream may comprise several contaminant compounds which are not the alcohol to be produced. The crude aldehyde stream therefore comprises at least one aldehyde and contaminant compounds. The crude alcohol stream therefore comprises at least one alcohol and contaminant compounds.

The term “contaminant” as used herein refers to undesirable non-alcohol compounds. Contaminants may include the products of side-reactions arising from hydroformylation reactions and/or hydrogenation reactions. For example, contaminant side-products formed from hydroformylation reactions may include the products of aldehyde condensation reactions (aldol reaction), esters, ketones, heterocycles, cycloalkanes, cycloalkenes, olefins, and the like. Particularly encountered contaminants include aldol condensation products and esters. Contaminant side-products formed from hydrogenation reactions may include catalyst residues, heterocycles, cycloalkanes, cycloalkenes, olefins, and the like. It is desirable to remove these contaminants from the purified alcohol stream which is the product.

As used herein, the term “heavy stream” refers to a fraction of organic compounds with a boiling point greater than the cut point (i.e. the boiling point range) for the alcohol to be purified. For instance, if the cut point for the alcohol to be purified is from 100 to 130° C., then the lowest boiling point for the heavy stream would be greater than 130° C. Those skilled in the art will appreciate that the lower limit of the boiling point range (lower limit of the cut point) will depend on the upper limit of the boiling point range for the at least one alcohol to be purified in the purified alcohol stream.

As used herein, the term “unsaturated” means compounds with carbon-carbon double bonds. “Saturated” compounds do not comprise carbon-carbon double bonds.

As used herein, the term “stripping gas” refers to a gas mixture capable of stripping light compounds such as alcohols and esters from a heavy stream comprising a mixture of organic compounds. The term ‘light’ within this context means the compounds comprised in the heavy stream with the lower boiling points relative to the rest of the compounds comprised in the heavy stream, and that are susceptible to being stripped from the heavy stream/vapourised when contacted with the stripping gas. Under the conditions in the stripping column, the stripping gas remains in the gaseous phase. The stripping gas is typically hydrogen.

As used herein, the term “stripping column” refers to a column configured to contact a stripping gas with a heavy stream to remove alcohol therefrom, and to provide a recycle stream comprising the stripping gas and the recovered alcohol. Those skilled in the art will be familiar with stripping columns. In embodiments, the stripping column is a hydrogen stripping column. In embodiments, the stripping column comprises sections to provide stages inside the column. Internals can be any shape or form that enables vapour-liquid contact and provide the number of stages required to strip the lighter compounds from the heavy stream. The stripping column may be provided with ‘internals’ arranged inside the column to provide said stages. In embodiments, the stripping column comprises 1-20 stages, optionally 1 to 8 stages. Internals may be selected, for example, from sieve trays, valve trays, bubble cap trays, random packing, or structured packing.

As used herein, the “corresponding” alcohol to an aldehyde is the reduced alcohol-form of that aldehyde. Inversely, the “corresponding” aldehyde to an alcohol is the oxidised aldehyde-form of that alcohol. For instance, those skilled in the art will appreciate that the “corresponding alcohol” to butanal is butanol. Within the context of the present invention, the corresponding alcohol of an unsaturated aldehyde is typically the corresponding saturated alcohol.

Those skilled in the art will appreciate that the desired aldehyde which provides the desired alcohol product can be obtained from hydroformylation of an olefin, wherein the olefin typically has one fewer carbon atoms than the corresponding desired aldehyde. For instance, those skilled in the art will appreciate than butanal can be obtained from hydroformylation of propene. Similarly, pentanal can be obtained from hydroformylation of butylene.

The methods described herein relate to the production of alcohol. In embodiments, at least one alcohol is produced, optionally at least two alcohols are produced. In typical embodiments, a particular alcohol is produced, also referred to herein as the ‘desired’ alcohol. In embodiments, the method according to the present invention is for the production of butanol. In other embodiments, the method according to the present invention is for the production of 2-ethyl hexanol. In other embodiments, the method according to the present invention is for the production of 2-propyl heptanol.

The crude alcohol stream according to the present invention comprises at least one alcohol, and may comprise a mixture of alcohols. Those skilled in the art will appreciate that fractional distillation may be used to isolate different alcohols based on their respective boiling points.

The crude alcohol stream may comprise a predominate alcohol, also referred to as the ‘desired’ alcohol. The crude alcohol stream may comprise greater than 50 wt % of the desired alcohol, relative to the total weight of the crude alcohol stream. In embodiments, the crude alcohol stream comprises greater than 60 wt % of the desired alcohol, and optionally greater than 70 wt % of the desired alcohol. In embodiments, the desired alcohol is selected from butanol, 2-ethyl hexanol, 2-propyl heptanol and isononyl alcohol.

The crude alcohol stream is distilled to provide a purified alcohol stream.

The purified alcohol stream may comprise greater than 95 wt % alcohol, optionally greater than 97 wt % alcohol, and optionally still greater than 99 wt % alcohol, relative to the total weight of the purified alcohol stream.

The purified alcohol fraction may contain a mixture of alcohols, but preferably comprises greater than 50 wt % of a desired alcohol. In preferred embodiments, the purified alcohol fraction comprises greater than 80 wt % of a desired alcohol, optionally greater than 95 wt % of a desired alcohol, optionally greater than 97 wt % of a desired alcohol, and optionally still greater than 99 wt % of a desired alcohol, relative to the total weight of the purified alcohol fraction. In some embodiments, for example when the desired alcohol is 2-propyl heptanol, the desired alcohol may be present as two or more isomers. In such embodiments, the purified alcohol fraction preferably comprises greater than 95 wt % of a desired alcohol and its isomers, optionally greater than 97 wt % of a desired alcohol and its isomers, and optionally still greater than 99 wt % of a desired alcohol and its isomers. In embodiments, the desired alcohol is selected from butanol, 2-ethyl hexanol and 2-propyl heptanol.

The distillation of the crude alcohol stream further provides a heavy stream. In embodiments, the heavy stream comprises less than 80 wt % alcohol, optionally less than 70 wt % alcohol, optionally less than 60 wt % alcohol. Preferably, the heavy stream comprises at least 40 wt %, preferably at least 50 wt % of alcohol. Those skilled in the art will appreciate that the term “heavy” as used herein refers to the fraction which has a higher boiling point that the purified alcohol stream. The heavy stream comprises contaminants. As already described above, it is desirable to avoid contaminants being present in the purified alcohol stream. For this reason, the cut point range of boiling temperatures used to isolate the purified alcohol stream is narrow. The effect of this is that significant amounts of alcohol may remain in the heavy stream. Having a high alcohol content in the heavy stream also advantageously suppresses the boiling temperature of the heavy stream and therefore reduces the bottoms temperature of a distillation column from which the heavy stream is withdrawn. High bottoms temperatures can lead to decomposition of heavy compounds in the distillation column to form light decomposition products and resulting contamination of the alcohol product stream by the light decomposition products.

It has been found that the alcohol present in the heavy stream may be recovered from said heavy stream, and reintroduced into the alcohol production process. In particular, the inventors have found that by contacting the heavy stream with a stripping gas, alcohol can be stripped from the heavy stream into a recycle stream which can be provided directly to the liquid phase hydrogenation reaction. In this way, the inventors have surprisingly discovered that from 50-90% of the alcohol in the heavy stream can be recovered, and typically from 60-80% of the alcohol in the heavy stream can be recovered. This represents significant improvements in the overall yield of alcohol recovered, relative to prior art methods where the alcohol in the heavy stream is not recovered. Unlike a vapour phase hydrogenation, where the aldehyde feed must be vaporised, a liquid phase hydrogenation does not have a feed vaporiser to which the heavy stream could be provided to recover the alcohol. The addition of a stripping column to a liquid hydrogenation system therefore represents a valuable addition to the process. Moreover, because the alcohol vaporised into the strip gas can recondense in the liquid phase hydrogenation reactor or prevent vaporisation of other alcohol in the liquid phase hydrogenation reactor by altering the vapour-liquid-equilibrium in the liquid phase hydrogenation reactor, more heat is made available to extract via the recycle cooler. In a vapour phase hydrogenation system, the temperatures are typically such that any heat must be transferred to cooling water. However, in a liquid phase hydrogenation system as in the present invention, the recycle cooler can operate at a temperature at which steam is raised and the extra heat resulting from the vaporised alcohol in the strip gas can therefore be recovered as useful steam resulting in an overall increase in energy efficiency compared to a prior art vapour phase hydrogenation process.

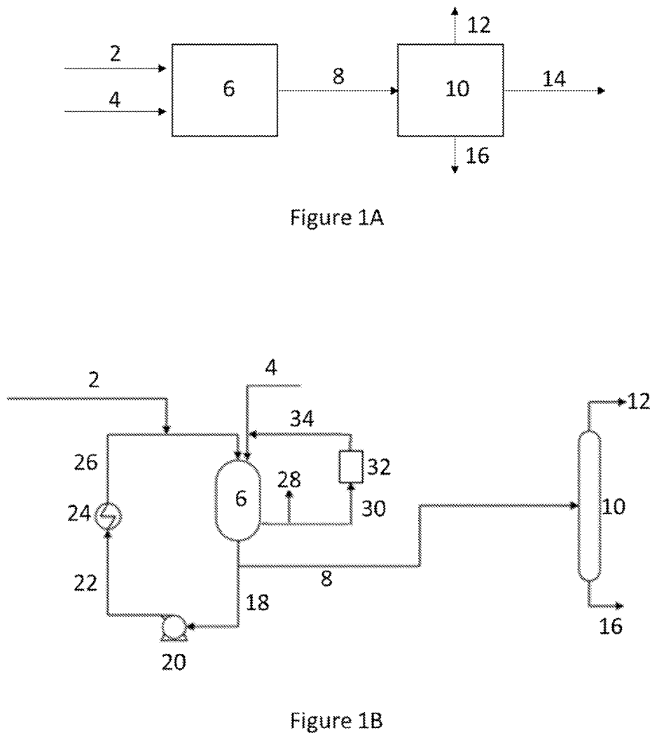

FIG. 1A is a schematic of a prior art method for the production of alcohol. A crude aldehyde stream comprising at least one aldehyde 2 together with a hydrogen stream 4 is provided to a hydrogenation reactor 6. The hydrogenation reactor is at a suitable temperature and pressure to effect hydrogenation of the at least one aldehyde to provide the corresponding alcohol in crude alcohol stream 8. The crude alcohol stream 8 is provided to distillation column 10, wherein the crude alcohol stream 8 is separated into a lights stream 12, purified alcohol stream 14 and heavy stream 16. In the prior art method depicted, the heavy stream 16 is disposed of.

FIG. 1B is a schematic of a prior art apparatus for implementing the method for the production of alcohol illustrated in FIG. 1. In FIG. 1B, the hydrogenation reactor 6 is a liquid phase hydrogenation reactor. The hydrogenation reactor is provided with a vent 28 to regulate the pressure in the reactor 6. Additionally, the hydrogen reactor is provided with a gas recycle compressor loop which may be used alternative to or in addition to the vent 28. The gas recycle compressor loop comprises an input stream 30 in fluid communication with a compressor 32, and an outlet stream 34 downstream of the compressor 32. The outlet stream 34 is in fluid communication with the hydrogen stream 4 which feeds into the hydrogenation reactor 6. Additionally, there is a provided a liquid phase recycling loop comprising a liquid recycle stream 18 downstream of the reactor 6 and in fluid communication with a pump 20, to provide a pumped liquid recycle stream 22. The liquid recycle stream 18 branches from the crude alcohol stream 8, although it may be alternatively drawn directly from the hydrogenation reactor 6. The pumped liquid recycle stream 22 is cooled in recycle cooler 24 to provide cooled pumped liquid recycle stream 26, before re-joining the crude aldehyde stream 2.

As described below, the methods and apparatus according to the invention and as described herein have several features in common with the prior art method and apparatus described above. Those skilled in the art will therefore appreciate that aspects of FIGS. 1A and 1B described above may be in common with the methods and apparatus according to the present invention, and the description of FIGS. 1A and 1B therefore also applies to the present invention insofar as there are common features.

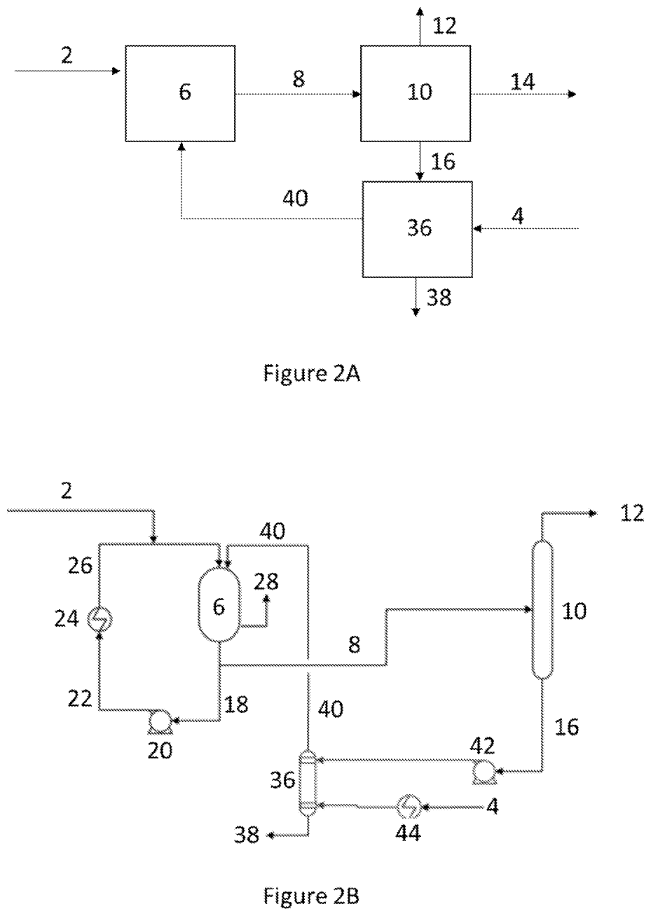

FIG. 2A is a schematic of a method for producing alcohol according to the present invention. FIG. 2A is identical to FIG. 1A, with the exception that the heavy stream 16 undergoes further processing and the hydrogen stream 4 is not directly provided to the hydrogenation reactor 6.

The crude aldehyde stream 2 may be provided as the effluent of a hydroformylation reaction (not illustrated). Hydroformylation provides aldehydes from olefins. Hydroformylation requires an olefin to be contacted with carbon monoxide and hydrogen over a catalyst (e.g. Cobalt- or Rhodium-based catalyst) to produce an aldehyde that contains 1 carbon more than the original olefin. The starting olefin material can therefore determine which alcohol is produced in the reaction process. For instance, hydroformylation of propylene gives a mixture of n-butanal and iso-butanal. Hydrogenation of n-butanal and iso-butanal provides 1-butanol and iso-butanol respectively. In addition, aldol condensation products of n-butanal such as 2-ethyl hexenal may also be provided, which when hydrogenated provides 2-ethyl hexanol. As another example, hydroformylation of propylene and butylene provides butanal and pentanal, which may be converted to 2-propyl hexenal by aldol condensation and hydrogenated to 2-propyl hexanol.

Suitable olefins for use in accordance with the present invention include propylene, butylene, pentene, hexene, heptene, octene, and isomers thereof. Particularly preferred olefins are selected from propylene and butylene. In embodiments, the crude aldehyde stream further comprises at least one aldol and/or at least one acrolein as the condensation products of at least two aldehydes. For example, aldol condensation of butanal (also referred to as butyraldehyde) with itself results in 2-ethyl-hexenal (also named ethyl-propyl-acrolein). In embodiments, suitable conditions are used in the hydroformylation reaction to promote aldol reactions between at least two aldehydes. However, during the hydroformylation synthesis several undesirable contaminants are produced and are present in the crude aldehyde stream.

Hydrogenation requires contacting the at least one aldehyde with hydrogen over a catalyst. Hydrogenation reduces Carbon-Oxygen double bonds to provide the corresponding Carbon-Oxygen single bond, and thus an alcohol. Hydrogenation may also reduce Carbon-Carbon double bonds, for instance where there at least one aldehyde in the crude aldehyde stream is unsaturated (e.g. an aldol condensation product). Hydrogenation may also cleave Carbon-Oxygen single bonds in esters to provide the corresponding alcohol. As such, the at least one aldehyde comprised in the crude aldehyde stream 2 may be saturated (i.e. does not contain carbon-carbon double bonds) or may be unsaturated (i.e. does contain carbon-carbon double bonds).

Catalysts used for use in the hydrogenation reactor are liquid phase hydrogenation reaction catalysts. The catalysts may be typically selected from Copper-, Copper/Zinc-, Copper/Chrome-, and Nickel-based catalysts.

During the hydroformylation and hydrogenation reactions several undesirable contaminants are produced that are comprised in the crude alcohol stream. Contaminants may include for instance esters. A particular contaminant ester formed in the production of butanol is, for example, butyl butyrate.

The crude alcohol stream 8 is provided as the effluent of the hydrogenation reaction. Distillation of the crude alcohol stream 8 is performed in distillation column 10 at temperatures and pressures effective to obtain the desired purified alcohol product stream 14 in the desired purity, and will be readily apparent to those skilled in the art.

In FIG. 2A, heavy stream 16 is directed to a stripping column 36, which is preferably a hydrogen stripping column. In contrast to FIGS. 1A and 1B, the hydrogen stream 4 is provided directly to the stripping column, instead of directly to hydrogenation reactor 6. The hydrogen stream 4 strips light components from the heavy stream 16, including alcohol which was not collected in the purified alcohol stream 14. As the hydrogen stream 4 strips lighter molecules (e.g. the at least one alcohol to be recovered) from the heavy stream (i.e. the lighter molecules transition from the liquid phase in the heavy stream to the vapour phase in the stripping gas), a recycle stream 40 is provided comprising hydrogen and the desired alcohol product, which is fed back into the hydrogenation reactor 6. The recycle stream 40 may comprise lighter molecules other than the at least one alcohol that were also stripped from the heavy stream. These other lighter molecules include esters and aldehydes which were not successfully reduced in the hydrogenation reactor 6, along with condensation products of aldehydes that were not successfully reduced in the hydrogenation reactor 6.

The waste stream 38 comprising the remaining heavy fraction compounds may be sent for further processing (not illustrated), such as cracking to further obtain useable aldehyde or alcohol molecules, or used as a fuel.

In the hydrogenation reactor 6, recycle stream 40 comprising hydrogen and the desired alcohol is contacted with fresh crude aldehyde stream 2 which may comprise at least one aldehyde. As already described above, the liquid phase hydrogenation occurs using a solid state catalyst to hydrogenate the aldehydes to the corresponding alkanols (saturated alcohols). As such, the recycle stream 40 returns otherwise wasted alcohol back into the process at the point of hydrogenation reactor 6, along with alcohol-precursor molecules such as aldehydes and esters.

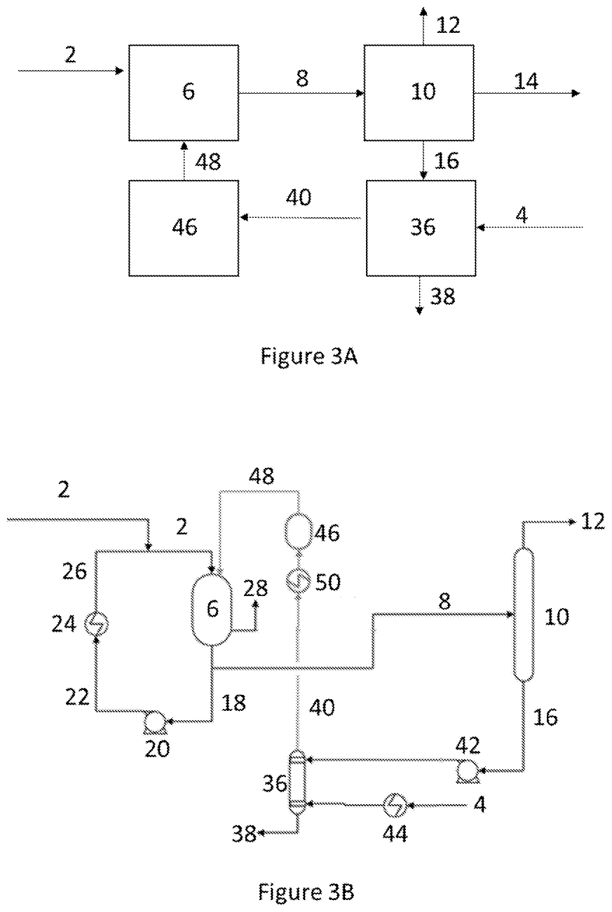

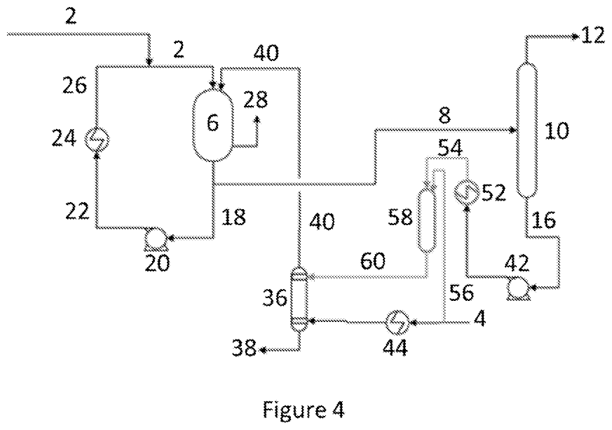

Although the stripping column 36 is described herein primarily with reference to stripping alcohols from a heavy stream 16, those skilled in the art will appreciate that other lighter molecule compounds may also be stripped and thus be present in the recycle stream 40, as mentioned above, such as aldehydes, esters and the like. That is particularly the case when the stripping column 36 is designed and operated so as to strip higher proportions of the alcohol from the heavy stream 16. For instance, in a process for the production of butanol, the hydrogen stripper may be designed and operated to recover most of the alcohol in the heavy stream 16 and may also therefore strip the ester butyl butyrate (BuBu), which forms as a side product in the production process. As the recycle stream 40 then comprises BuBu, the process illustrated in FIG. 2A and the apparatus illustrated in FIG. 2B is likely to create an accumulation of BuBu. Although BuBu may be hydrogenated to butanol in the hydrogenation reactor 6, there may be insufficient conversion therein. For this reason, it may be desirable for the recycle stream 40 to undergo further treatment to remove contaminants such as BuBu, prior to the recycle stream 40 entering the hydrogenation reactor 6 (see FIGS. 3A, 3B and 4). That may increase alcohol yield both by converting at least some of the contaminants to further alcohol product and by facilitating the design and operation of the stripping column 16 so as to maximise recovery of alcohol from the heavy stream 16.

FIG. 2B is a schematic of an apparatus suitable for implementing the method depicted in FIG. 2A. FIG. 2B is identical to FIG. 1B with the exception heavy stream 16 is subject to further processing as described below, the hydrogen stream 4 is not provided directly to the hydrogenation reactor 6, and the (optional) gas recycle compressor loop is absent.

As shown in FIG. 2B, heavy stream 16 is directed to the top of a stripper column 36. The heavy stream 16 may be pumped into the stripper column 36 by means of a pump 42. The hydrogen stream 4 is directed to the bottom of the stripper column 36, and is optionally heated using heater 44. The heated hydrogen stream 4 rises within the stripper column 36 and contacts the descending heavy stream 16 to strip the alcohol from said heavy stream. This enables at least a portion of the lighter components (e.g. alcohol product) in the heavy stream to migrate from the liquid phase in the heavy stream to the vapour phase in the hydrogen stream. Ideally only the lighter components (e.g. alcohol product) are evaporated and the heavier components remain in the liquid phase. Advantageously, the extent of the vaporisation of lighter components (e.g. alcohol) from the heavy stream can be controlled by adjusting the temperature of the hydrogen stream 4 fed into the hydrogen stripper column 36, adjusting the temperature of the heavy stream 16 and/or bypassing a proportion of the hydrogen stream 4 around the stripping column 36. For example, if the hydrogen stream 4 temperature is raised, a bigger fraction of the heavy steam will be evaporated. The recycle stream 40 comprising hydrogen and the recovered alcohol is extracted from the top of the stripper column, and directed to the hydrogenation reactor 6 as previously described.

The waste stream 38 is extracted from the bottom of the stripper column. The waste stream 38 may be further distilled, or undergo a cracking process to obtain useful smaller-carbon molecules such as saturated and unsaturated aldehydes, enols, acetals, and the like. These products may be used to create valuable secondary products or can be re-inserted in the process to contribute to the yield of the alcohol product.

In embodiments, the operating pressure inside the distillation column 10 is less than the operating pressure inside the hydrogenation reactor 6, and the operating pressure inside the stripper column 36 is higher than the hydrogenation reactor 6. The operating pressure of the stripper column 36 being greater means a flow of recycle stream 40 is provided to the hydrogenation reactor 6 without the need for a compressor. Alternatively, a compressor may be provided in the flow path of recycle stream 40.

The inclusion of a gas makeup to the hydrogen stripper can assist the stripper or reduce the temperature in the preheater.

The provision of vent 28 and gas recycle compressor loop (30, 32, 34) as described above in relation to FIG. 1B, are each optional features of the present invention. The vent 28 operates to vent inerts from the liquid phase hydrogenation reactor 6 and prevent their build up. The vent will also contain some hydrogen. The hydrogen 4 entering the reactor is readily consumed in the hydrogenation reactor, and may be adsorbed into the liquid phase therein. If hydrogen consumption is reduced, and hydrogen remains in the gaseous phase in the reactor, it may be vented via vent 28, or compressed for re-use via a gas recycle compressor loop. This may be particularly beneficial when a secondary vapour phase hydrogenation reactor (as described for example in relation to FIG. 3B below) is used. The skilled person will appreciate that the gas recycle compressor loop does not provide hydrogen to the liquid phase hydrogenation reactor 6 to effect a vapour phase hydrogenation reaction therein because the crude aldehyde- and crude-alcohol stream remain in the liquid phase and the reaction in liquid phase hydrogenation reactor 6 is therefore a liquid phase hydrogenation reaction.

The liquid phase recycling loop (18, 20, 22, 24, 26), also described above in relation to FIG. 1B, and particularly the recycle cooler 24, may combine with the other features of the invention to produce an advantageous energy efficiency compared to prior art vapour phase processes. The hydrogenation reactor 6, which is a liquid phase hydrogenation reactor comprises a solid catalyst, and the temperature and pressure therein is such that the crude aldehyde stream and the crude alcohol stream produced remains in the liquid form. The liquid phase recycling loop operates to cool the crude aldehyde stream. Because the alcohol in the recycle stream 40 is in the vapour phase, it will condense, or affect the vapour-liquid equilibrium such that other alcohol does not vaporise, in the liquid phase hydrogenation reactor 6. As a result, at least some of the energy used to vaporise the alcohol in the stripping column 36 is effectively transferred to the liquid phase in the liquid phase hydrogenation reactor 6 and can be recovered again in the recycle cooler 24. Because the recycle cooler 24 recovers usable heat, for example by operating at a temperature suitable for raising steam, that energy is recovered in a usable form and not just lost to cooling water as would be the case in prior art vapour phase processes. The additional energy cost of the stripping column 26 is therefore reduced by its use in combination with the liquid phase recycle loop and in particular the recycle cooler 24.

FIG. 3A is a schematic of a method for producing alcohol according to the present invention. FIG. 3A is identical to FIG. 2A described above, with the exception that the recycle stream 40 undergoes further processing as described below.

FIG. 3B is a schematic of an apparatus suitable for implementing the method depicted in FIG. 3A. FIG. 3B is identical to FIG. 2B with the exception recycle stream 40 is subject to further processing as described below.

Recycle stream 40 is directed to heater 50, and then to a vapour phase hydrogenation reactor 46 to provide processed stream 48. The purpose of the vapour phase hydrogenation reactor 46 is to cleave esters present in the recycle stream 40. For instance, the vapour phase hydrogenation reactor may cleave the ester butyl butyrate to provide butanol, thus increasing the yield of alcohol product in the processed stream 48 that is returned to the reaction process. Advantageously, treating the recycle stream 40 in this way can prevent accumulation of heavy contaminant esters such as butyl butyrate in the main hydrogenation reactor 6, which in the liquid phase could hinder efficient hydrogenation of the crude aldehyde feedstock. It may also advantageously allow the stripping column 36 to be operated so as to maximise alcohol recovery from the heavy stream 16 without causing accumulation of contaminants such as those esters.

FIG. 4 is a schematic of an apparatus suitable for implementing the methods of the present invention. FIG. 4 is identical to FIG. 2B with the exception the heavy stream 16 is subject to further processing as described below.

Downstream of pump 42, the heavy stream 16 is directed to a heater 52 to provide a heated heavy stream 54 which is then directed to a liquid phase hydrogenation reactor 58. The liquid phase hydrogenation reactor 58 provides a processed stream 60 which advantageously comprises fewer contaminants such as esters (e.g. butyl butyrate) and the like as compared to the untreated heavy stream 16 of FIG. 2B. The processed stream 60 is then directed to the stripper column 36, as described above in relation to FIG. 2B. The liquid phase hydrogenation reactor 58 advantageously hydrogenates contaminants such as esters into useful products such as the desired alcohol, thus improving and increasing the overall yield of product obtained.

### EXAMPLES

### Example 1

### High Pressure Hydrogen Stripper in Liquid Phase Hydrogenation (LPH) Loop for Butanol

A heavy stream (flow rate 530 kg/h) from distillation of butanol, comprising approximately 41 wt % Butanol, 44 wt % C8 oxygenates, and 15 wt % C12 oxygenates, was supplied to a hydrogen stripper column at 137° C. The pressure in the hydrogen stripper column was 32 bara in the top. A stripping gas (hydrogen) stream (flow rate 1294 kg/h) comprising approximately 99 mol % hydrogen and 1 mol % methane was heated to 75° C. and supplied to the bottom of the hydrogen stripper column. The hydrogen stripper column comprised internals to provide 6 theoretical trays.

A liquid waste stream (flow rate 350 kg/h) at 71° C. comprising approximately 22 wt % butanol, 56 wt % C8 oxygenates and 22 wt % C12 oxygenates was obtained from the bottom the hydrogen stripper.

A recycle (hydrogen-rich vapour) stream (flow rate 1476 kg/h) at 75° C. comprising approximately 81 wt % hydrogen, 6.5 wt % methane, 9.5 wt % butanol and 2.7 wt % C8 oxygenates (and trace amounts of C12 oxygenates) was obtained from the top of the hydrogen stripper.

The recycle stream comprising butanol was fed to a liquid phase hydrogenation reactor, upstream of the distillation column used to provide the heavy stream.

In this example 64.5% of butanol was recovered from the heavy stream and returned to the process.

### Example 2

### High Pressure Hydrogen Stripper in Liquid Phase Hydrogenation (LPH) Loop for 2-EthylHexanol

A heavy stream (flow rate 644 kg/h) from distillation of 2-Ethylhexanol (2EH), comprising approximately 15.5 wt % 2EH, 75 wt % C12 oxygenates, and 9 wt % C16 oxygenates, was supplied to a hydrogen stripper column at 152° C. The pressure of the hydrogen stripper column was 32 bara in the top. A stripping gas (hydrogen) stream (flow rate 467 kg/h) comprising approximately 98 mol % hydrogen and 2 mol % methane was heated to 160° C. and supplied to the bottom of the hydrogen stripper column. The hydrogen stripper column comprised internals to provide 6 theoretical trays.

A liquid waste stream (flow rate 550 kg/h) at 156° C. comprising approximately 5 wt % 2EH, 85 wt % C12 oxygenates and 10 wt % C16 oxygenates was obtained from the bottom the hydrogen stripper.

A recycle (hydrogen-rich vapour) stream (flow rate 560 kg/h) at 154° C. comprising approximately 71.7 wt % hydrogen, 11.6 wt % methane, 12.8 wt % 2EH, 3.3 wt % C12 oxygenates and 0.4 wt % C16 oxygenates was obtained at the top of the hydrogen stripper.

The recycle stream comprising 2EH was fed to a liquid phase hydrogenation reactor, upstream of the distillation column used to provide the heavy stream.

In this example 72% of 2EH is recovered from the heavies draw and returned to the process.

1. A method for the production of at least one alcohol comprising:
(i) providing a crude aldehyde stream comprising at least one aldehyde to at least one liquid phase hydrogenation reactor, and hydrogenating the at least one aldehyde in a liquid phase hydrogenation reaction to provide a crude alcohol stream comprising at least one alcohol;
(ii) recycling a liquid recycle stream comprising the at least one alcohol to the at least one liquid hydrogenation reactor via a recycle cooler in which heat in the liquid recycle stream is recovered;
(iii) providing the crude alcohol stream to a distillation column and performing distillation on the crude alcohol stream to obtain a purified alcohol stream including the at least one alcohol, and a heavy stream including the at least one alcohol;
(iv) providing the heavy stream to a stripping column and contacting the heavy stream with a stripping gas comprising at least 20 mol % hydrogen to separate the heavy stream into a recycle stream including the at least one alcohol, and a waste stream;
(v) returning at least a portion of the recycle stream to the at least one liquid phase hydrogenation reactor in step (i).

2. The method according to claim 1, wherein the at least one alcohol is at least one C₃-C₂₀ alcohol, optionally at least one C₃-C₁₀ alcohol.

3. The method according to claim 2, wherein the at least one alcohol is selected from butanol, 2-ethyhexanol, 2-propyl heptanol and isononyl alcohol.

4. The method according to claim 1, wherein the crude alcohol stream comprises at least 50 wt % of the at least one alcohol, optionally at least 60 wt % of the at least one alcohol, optionally still at least 70 wt % of the at least one alcohol.

5. The method according to claim 1 wherein the purified alcohol stream comprises greater than 95 wt % of the at least one alcohol, optionally greater than 97 wt % of the at least one alcohol, and optionally greater than 99 wt % of the at least one alcohol, relative to the total weight of the purified alcohol stream.

6. The method according to claim 1 wherein the heavy stream comprises less than 80 wt % of the at least one alcohol, optionally less than 70 wt % of the at least one alcohol, optionally less than 60 wt % of the at least one alcohol.

7. The method according to claim 1, wherein the stripping gas stream comprises at least 70 mol % hydrogen, optionally at least 90 mol % hydrogen, optionally still at least 98 mol % hydrogen.

8. The method according to claim 7 wherein the recycle stream comprises at least 60 wt % hydrogen, optionally at least 70 wt % hydrogen, optionally still at least 80 wt % hydrogen.

9. The method according to claim 7 wherein the recycle stream comprises at least 70 wt % hydrogen and at least 5 wt % alcohol.

10. The method according to claim 1, wherein the pressure in the stripping column is from 15-45 bara, optionally from 20-35 bara.

11. The method according to claim 1:
(i) wherein the stripping gas stream is provided to a lower portion of the stripping column, optionally to the bottom of the stripping column; and
(ii) wherein the heavy stream is provided to an upper portion of the stripping column, optionally to the top of the stripping column.

12. The method according to claim 11 wherein the recycle stream is obtained from an upper portion of the stripping column, optionally from the top of the stripping column.

13. The method according to claim 1, wherein the temperature of the stripping gas stream entering the stripping column is from 50250° C., optionally from 60-200° C.

14. The method according to claim 1, wherein the temperature of the heavy stream entering the stripping column is from 50-300° C., optionally from 100-200° C., optionally from 125-175° C.

15. The method according to claim 1, wherein the step of providing a crude aldehyde stream comprises the steps of:
providing an olefin stream to a hydroformylation reactor, and contacting the olefin stream with a hydroformylation gas stream comprising hydrogen and carbon monoxide to effect a hydroformylation reaction to provide the crude aldehyde stream.

16. The method according to claim 1, wherein the crude aldehyde stream comprises at least one C₃-C₂₀ aldehyde, optionally at least one C₃-C₁₀ aldehyde, optionally at least one C₃-C₈ aldehyde.

17. The method according to claim 16, wherein the at least one aldehyde is selected from butanal, 2-ethyl hexenal, 2-propyl-heptenal and iso nonanal.

18. The method according to claim 15, wherein providing the crude aldehyde stream further comprises subjecting a reaction effluent of the hydroformylation reaction to an aldol reaction between at least two aldehydes comprised in the reaction effluent, to provide the crude aldehyde stream.

19. The method according to claim 1, wherein prior to step (v) the recycle stream is directed to a secondary hydrogenation reactor configured to hydrogenate esters into at least one alcohol.

20. The method according to claim 19 wherein the hydrogenation reaction in the secondary hydrogenation reactor is a vapour phase hydrogenation reaction or a liquid phase hydrogenation reaction.

21. An apparatus for the production of alcohol comprising:
at least one liquid phase hydrogenation reactor configured to hydrogenate at least one aldehyde comprised in a crude aldehyde stream in a liquid phase hydrogenation reaction to provide a crude alcohol stream comprising at least one alcohol;
a recycle loop in fluid communication with the at least one liquid phase hydrogenation reactor and a recycle cooler, the recycle loop being configured to recycle a liquid recycle stream from the at least one liquid phase hydrogenation reactor, via the recycle cooler, to the at least one liquid phase hydrogenation reactor;
a distillation column in fluid communication with the at least one liquid phase hydrogenation reactor, the distillation column being configured to perform distillation on the crude alcohol stream to provide a purified alcohol stream comprising the at least one alcohol, and a heavy stream comprising the at least one alcohol;
a stripping column in fluid communication with the distillation column, the stripping column being configured to contact said heavy stream with a stripping gas stream comprising at least 20 mol % hydrogen to separate the heavy stream into a recycle stream comprising the at least one alcohol, and a waste stream;
wherein the stripping column is in fluid communication with the at least one liquid phase hydrogenation reactor in step (i) to provide at least a portion of the recycle stream to the at least one liquid phase hydrogenation reactor.

22. The apparatus according to claim 21 further comprising:
a hydroformylation reactor configured to contact an olefin stream comprising at least one olefin with hydrogen and carbon monoxide to effect a hydroformylation reaction on the at least one olefin, to provide the crude aldehyde stream;
wherein the at least one liquid phase hydrogenation reactor is in fluid communication with the hydroformylation reactor.
